(19) United States
(12) Patent Application Publication
(10) Pub. No.: US 2015/0351676 A1
(43) Pub. Date: Dec. 10, 2015

(54) Automatic Blood Collection

(71) Applicant: LabATM, Inc., Solana Beach, CA (US)

(72) Inventors: Philippe Faurie, Solana Beach, CA (US); Diyana Ratnashanti Gunawardena, Solon, OH (US); Rajan Ramaswamy, San Diego, CA (US); Nathan Balcom, Oceanside, CA (US)

(21) Appl. No.: 14/301,289

(22) Filed: Jun. 10, 2014

(51) Int. Cl. A61B 5/151; A61F 13/00; A61B 5/15

(52) U.S. Cl. CPC A61B 5/15186; A61B 5/1411; A61B 5/150076; A61B 5/150099; A61B 5/150068; A61B 5/150312; A61B 5/150343; A61B 5/150748; A61F 13/00085

(57) ABSTRACT

A self-contained universal front-end automatic blood collection device is disclosed herein. The device collects a few micro-liters blood sample that is used for a broad range of blood analyzers. The design of the front-end closely mimics current phlebotomist practice and provides a number of unique features that ensure patient safety and sterility, improve collection efficacy, and prevent sample contamination and user cross contamination.

### CROSS REFERENCE TO RELATED APPLICATION

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention generally relates to blood collection. More specifically, the present invention relates to automatic blood collection.

2. Description of the Related Art

The prior art discusses various devices to collect blood from a patient.

Currently when collection of blood with a capillary tube is required for finger-stick testing the procedure involves a six step process that typically requires a phlebotomist (a person skilled in the process of making an incision in a vein with a needle). This process is 1—cleaning the finger 2—pricking the finger with a commercially available lancet that contains a clean needle, 3—wiping off of the first droplet of blood, 4—using a capillary tube to collect the quantity of blood needed for testing, 5 transport the blood into a blood analyzer device, 6 Applying a bandage around finger at the puncture location, and 7 dispose all the contaminated materials in a safe waste.

### BRIEF SUMMARY OF THE INVENTION

The present invention provides a solution to blood collection.

One aspect of the present invention is a self-contained universal front-end automatic blood collection device that collects a few micro-liters blood sample that is used for a broad range of blood analyzers. The design of the front-end closely mimics current phlebotomist practice and provides a number of unique features that ensure patient safety and sterility, improve collection efficacy, prevent sample contamination and user cross contamination. All the features are integrated into a compact, low-cost package.

Another aspect of the present invention is an apparatus for automatic blood collection from a patient. The apparatus comprises a housing having an insertion port, a lancet comprising a needle, a dispenser positioned within the housing, a mechanism for removing an initial droplet of blood, a capillary tube for collecting a predetermined amount of blood, and a dressing dispenser. The lancet is positioned within the housing and in communication with the insertion port. The dispenser comprises a sanitizing agent. The mechanism removing an initial droplet of blood is positioned within the housing. The dressing dispenser is located at an opening of the insertion port. The dressing dispenser comprises a bandage. The capillary tube is positioned within the housing and in communication with the insertion port. The dispenser is adapted to dispense the sanitizing agent on a portion of a finger of a patient placed within the insertion port. The needle is adapted to pierce the sanitized finger. The mechanism is adapted to remove the initial droplet of blood from the pierced finger. The capillary is adapted to collect the predetermined amount of blood from the patient. The dressing dispenser is adapted to wrap the bandage around the pierced finger of the patient.

Yet another aspect of the present invention is a method for automatic blood collection from a patient. The method includes inserting a finger of a patient into a port of an automatic blood collection apparatus. The method also includes sanitizing a portion of the finger of the patient within the automatic blood collection apparatus. The method also includes piercing the sanitized portion of the finger with a needle of a lancet. The method also includes removing a first droplet of blood from the pierced finger. The method also includes collecting a predetermined amount of blood from the pierced finger in a capillary tube. The method also includes transporting the predetermined amount of blood to a blood analyzer device. The method also includes bandaging the pierced finger of the patient with a wrap within the automatic blood collection apparatus. The method also includes disposing of a plurality of contaminated materials from the automatic blood collection apparatus.

Yet another aspect of the present invention is an apparatus for dressing a punctured finger of a patient subsequent to automatic blood collection from a patient. The apparatus comprises a housing having an insertion port, and a dressing dispenser located at an opening of the insertion port. The dressing dispenser comprises a dressing. The dressing dispenser is adapted to automatically wrap the dressing around a pierced finger of the patient as the pierced finger is removed from the insertion port. The dressing protects and avoids cross contamination of the finger.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a block diagram of automatic blood collection.

FIG. 2 is an illustration of the disposable components and the control components for automatic blood collection.

FIG. 2A is an exploded view of an automatic blood collection apparatus.

FIG. 3 is an illustration of an automatic blood collection apparatus within an opened sterile package.

FIG. 4 is an illustration of a patient inserting a finger into an automatic blood collection apparatus.

FIG. 5A is a side view of an automatic blood collection apparatus.

FIG. 5B is a front view of an automatic blood collection apparatus.

FIG. 5C is an opposing side view of an automatic blood collection apparatus.

FIG. 5D is a rear view of an automatic blood collection apparatus.

FIG. 6 is a cross-sectional view of an automatic blood collection apparatus.

FIG. 6A is cross-sectional view of an automatic blood collection apparatus with a patient's finger inserted therein.

FIG. 7 is an illustration of a patient finger entering an automatic blood collection apparatus and a cross sectional view of the blood collection receptacle of FIG. 6A.

FIG. 7A is an illustration of sanitizing a patient finger and a cross sectional view of the blood collection receptacle of FIG. 6A.

FIG. 7B is an illustration of piercing a patient finger and a cross sectional view of the blood collection receptacle of FIG. 6A.

FIG. 7C is an illustration of blood collection from a patient finger and a cross sectional view of the blood collection receptacle of FIG. 6A.

FIG. 8 is a flow chart a method for automatic blood collection from a patient.

FIG. 9 is an illustration of a patient at a kiosk for automatic blood collection.

### DETAILED DESCRIPTION OF THE INVENTION

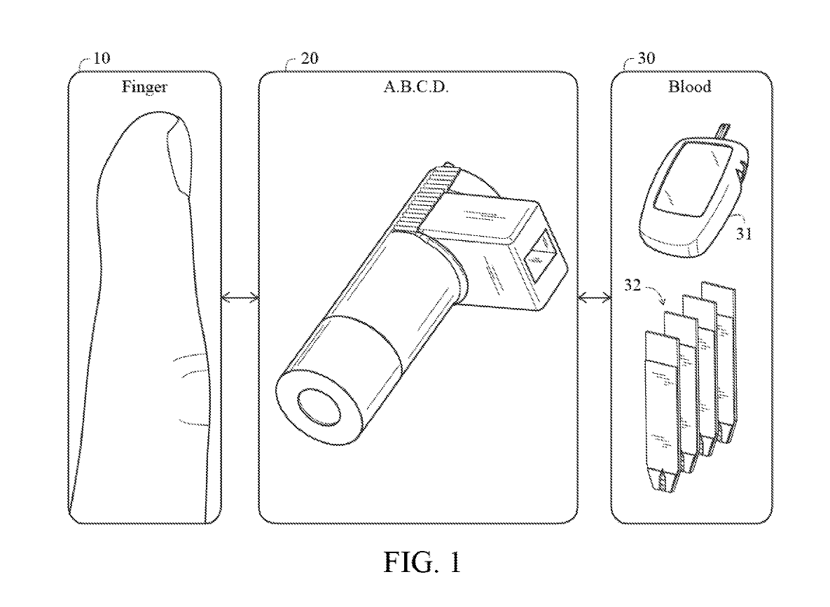

As shown in FIG. 1, the automatic blood collection apparatus 20 is interfaced on one side with a patient's finger 10, and on the other side with a broad range of blood related components 30, such as blood analyzers 31 or blood test strips 32.

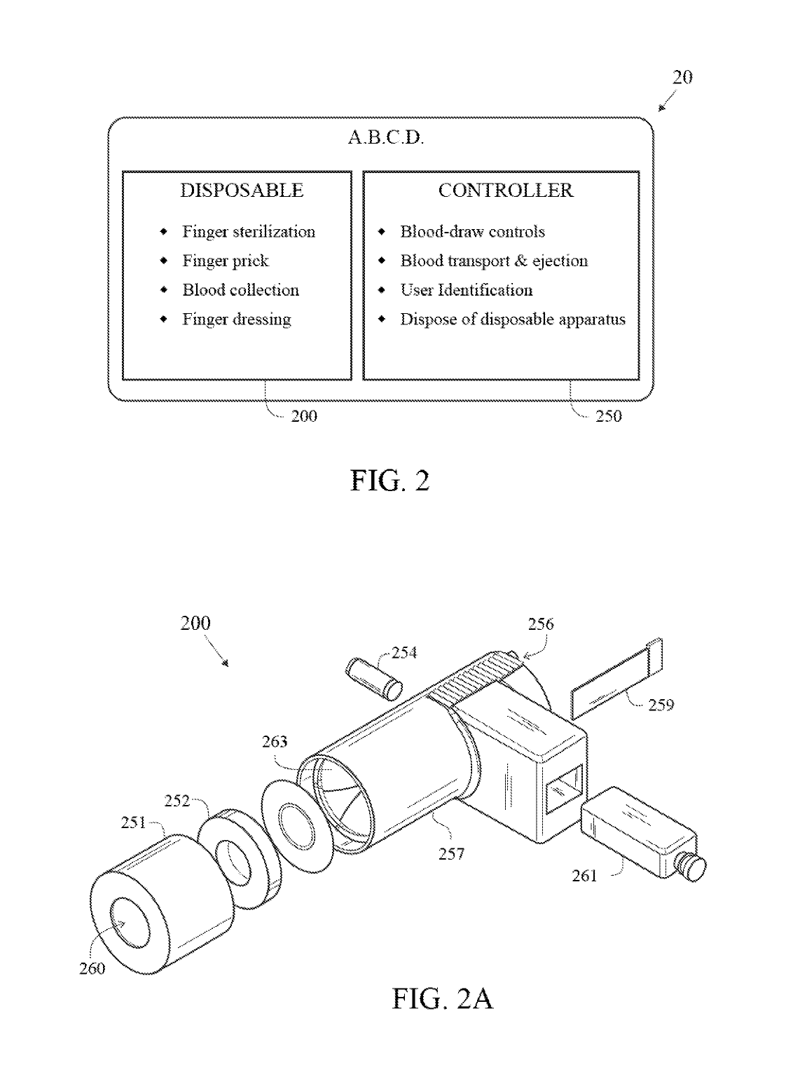

As illustrated in FIG. 2, the automatic blood collection apparatus 20 is utilized with disposable components 200 and controller components 250. The disposable components 200 preferably comprise a finger sterilization component, a finger prick component, a blood collection component and a finger-dressing component. The controller component 250 preferably comprises blood draw control components, blood transport and ejection components, user identification components and disposal components.

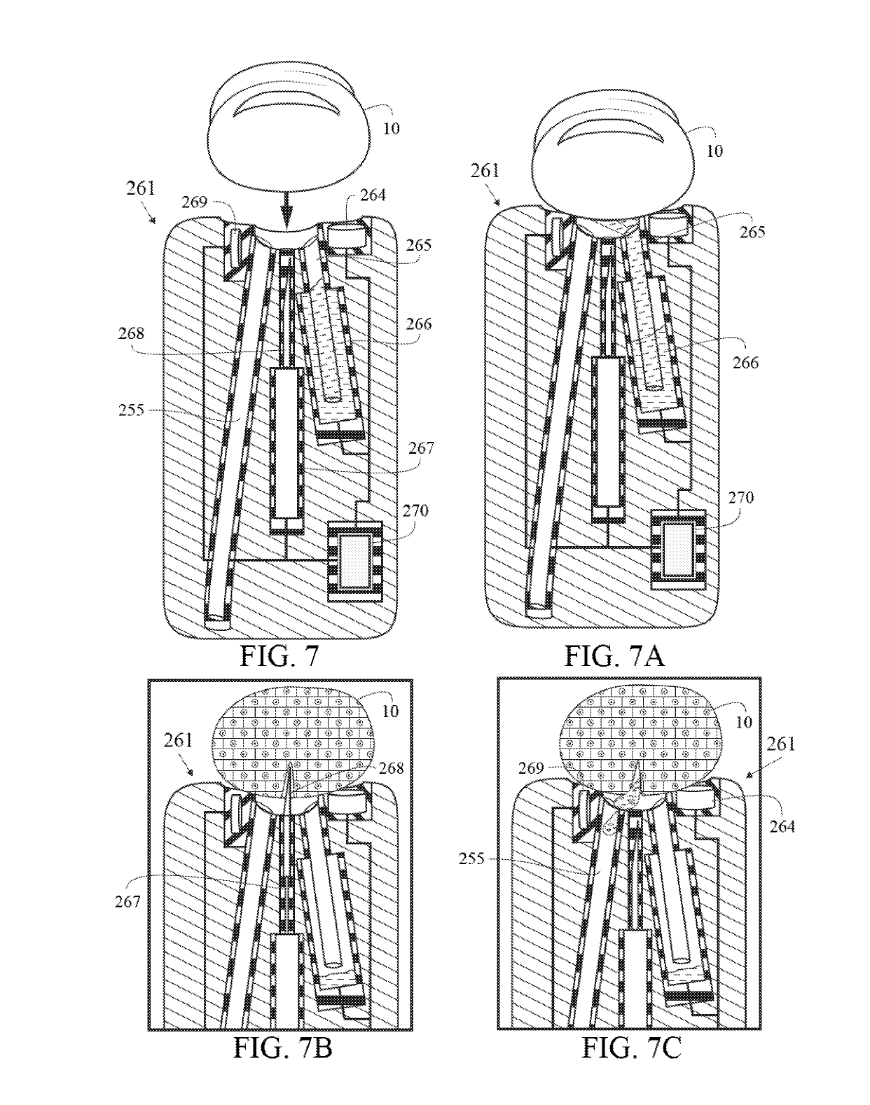

As shown in FIG. 2A, the automatic blood collection apparatus 200 preferably comprises a main body/housing 257 of the apparatus, an insertion port 260 for receiving a finger of a patient, a lancet having a puncturing needle, an actuator 254 which biases the patient's finger towards a puncturing needle, a vacuum port 256 to aid in blood withdrawal, a strip 259 for removing a first drop of blood, a capillary tube for collecting a predetermined amount of blood, a blood collection receptacle for receiving the blood from the capillary tube, a dressing mechanism 251 comprising a bandage 252 for bandaging a punctured finger, and a disposable container 261 for containing a needle and blood collection components. The automatic blood collection apparatus 200 may also include massaging mechanism 263 having nubs for messaging a patient finger in order to increase the flow of blood, and other optional components as shown in FIGS. 7-7C.

The housing 257 preferably composed of a sterile material such as a polycarbonate plastic material, an ABS material, a stainless steel material, or the like.

The insertion port 260 preferably has a length ranging from 2 centimeters to 10 centimeters, more preferably from 4 centimeters to 8 centimeters, and most preferably 6 centimeters. A diameter of the insertion port preferably ranges from 1 centimeter to 3 centimeters, and is most preferably 2 centimeters. The biasing member/actuator 254 aligns a patient's finger in an optimal position within the insertion port 262 with the puncturing needle for blood collection.

The lancet is preferably positioned near the internal end of the insertion port 262. The puncturing needle is preferably the type typically utilized by a phlebotomist such as a 21 g (green top) needle, a 22 g (black top) needle, a 21 g (green label) butterfly needle, a 23 g (blue label) butterfly needle, and a 25 g (orange label) butterfly needle.

In a preferred embodiment, a transport mechanism transports the blood collection receptacle containing blood to a blood analyzer. The blood collection receptacle is preferably a vacuum tube such as a VACUTAINER tube available from Becton, Dickinson & Company, or a similar generic model. Preferably, the blood collection receptacle contains up to ten milliliters of blood, and alternatively contains from 1 milliliter to 10 milliliters of blood from the patient.

In a preferred embodiment, the apparatus 200 further comprises a heater adapted to warm the finger within the automatic blood collection apparatus 200 to enhance a blood flow of the patient.

In a preferred embodiment, the apparatus 200 further comprises a massage mechanism adapted to massage the patient's finger within the automatic blood collection apparatus 200 to enhance a blood flow of the patient.

In a preferred embodiment, the apparatus 200 further comprises a vacuum mechanism to withdraw air from the insertion port of the automatic blood collection apparatus 200 to enhance a blood flow of the patient. The vacuum pressure ranges from 0 to negative 600 mmHg. Negative pressure signifies pressure less than standard atmospheric pressure (14.7 phi or 760 mm Hg at sea level). A vacuum pump is placed in flow communication with the insertion port in order to create the vacuum or partial vacuum in the insertion port for assistance in the flow of blood from the patient.

In a preferred embodiment, the apparatus 200 further comprises a sensor for measuring the amount of blood drawn from the patient. The sensor is positioned within the housing 257.

In a preferred embodiment, the apparatus 200 further comprises an ejection mechanism for ejecting the blood collection receptacle from the housing 257.

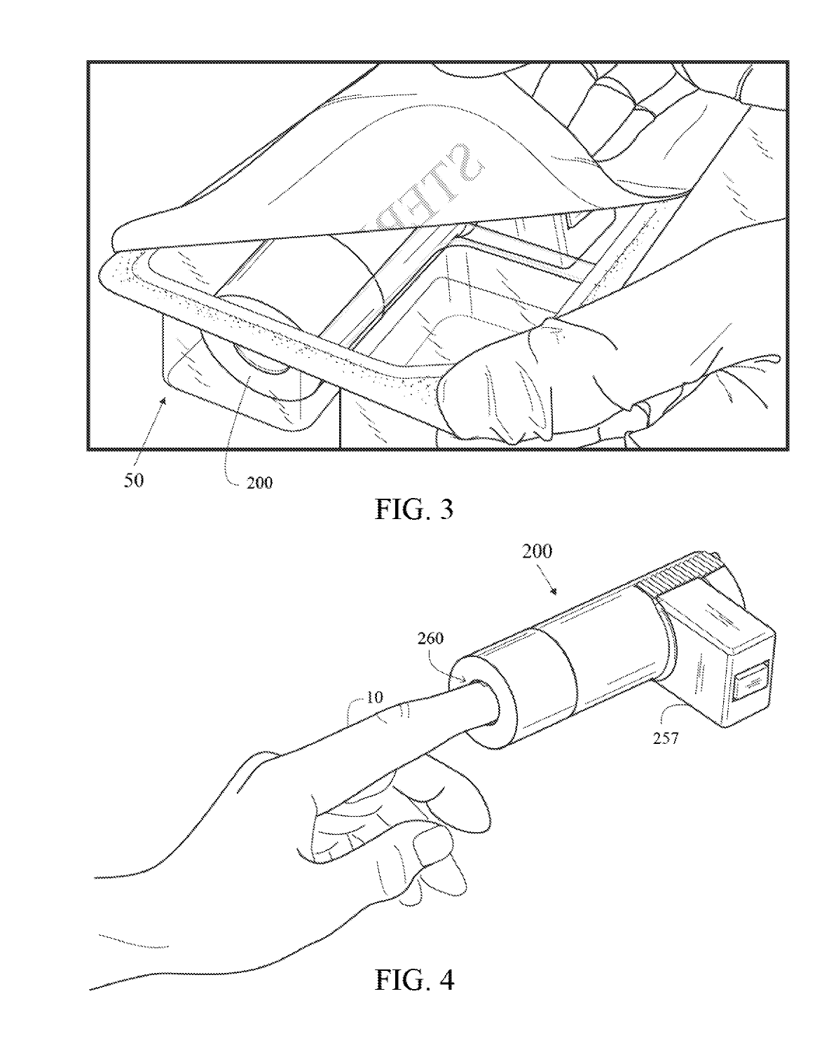

As illustrated in FIG. 3, the disposable apparatus 200 is preferably enclosed in a sterile package 50 prior to initial use. In this manner, a patient is provided with greater certainty that the apparatus 200 is safe for use in collecting blood from the patient.

FIG. 4 shows the placement of a user's finger 10 within the automatic blood collection apparatus 200 after removing the apparatus 200 from the sterile packaging 50.

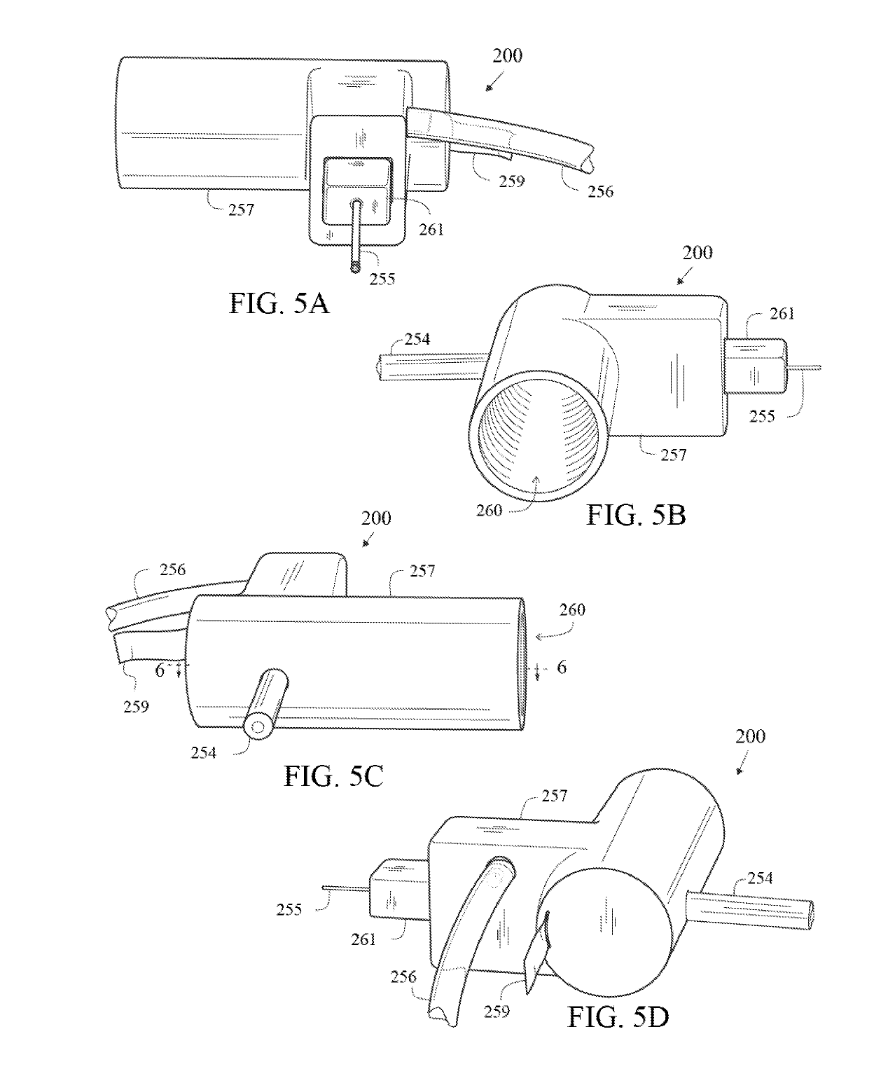

FIGS. 5A-5D illustrate another embodiment of the disposable apparatus 200 from different perspective views showing the different components of the apparatus 200. FIG. 5A is a side view showing a main body of the apparatus 257, a capillary tube 255, a disposable container 261, a vacuum port with tubing 256, and a strip 259 for removing the first drop of blood. FIG. 5B is a front view showing a main body of the apparatus 257 with the insertion port 260, a capillary tube 255 extending from a disposable container 261, and an actuator 254. FIG. 5C is an opposing side view showing an actuator 254, a strip 259 for removing the first drop of blood, a vacuum port with tubing 256, and a main body of the apparatus 257. FIG. 5D is a rear view showing a main body of the apparatus 257, a capillary tube 255, a disposable container 261, a vacuum port with tubing 256, a strip 259 for removing the first drop of blood, and an actuator 254.

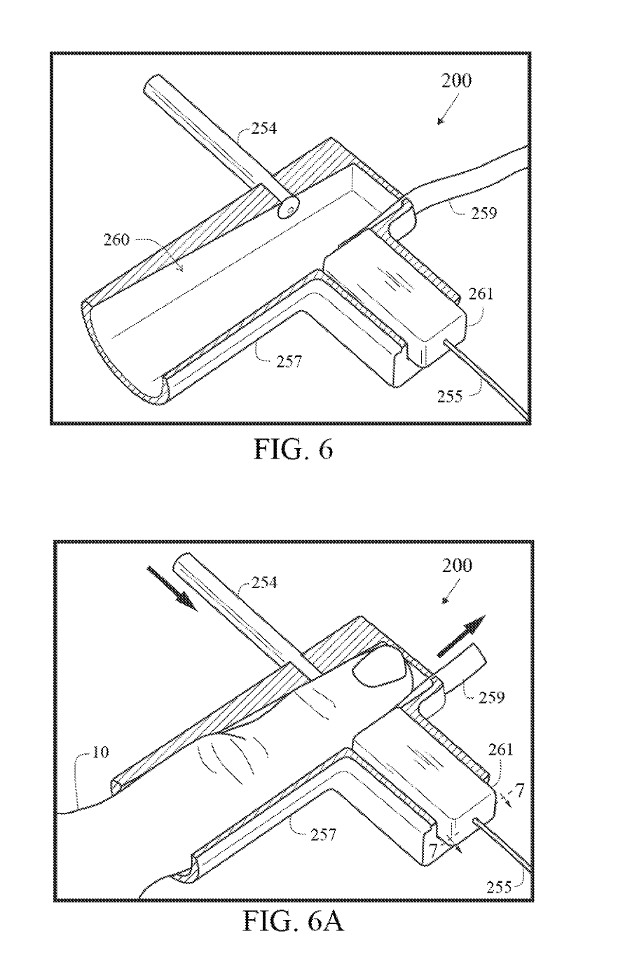

FIGS. 6-6A are sectional views of the disposable apparatus 200. FIG. 6A illustrates the placement of a user's finger 10 within the apparatus 200. As the actuator 254 is pushed downward, towards the finger 10, the needle contained within the disposable container 261 pricks the finger 10 and the strip 259 is pulled outwards to remove the first drop of blood.

FIG. 7 illustrates the automatic blood collection apparatus 20 utilized by a patient 15. The disposable apparatus 200 is inserted into the controller apparatus 250. The controller apparatus 250 comprises one or more of the following elements: a control display screen 246; a bay 245, including a mechanical lock (not shown), wherein the disposable apparatus 200 is locked into place; biometric identification components, such as a camera 249, a fingerprint reader 248, or an identification card reader 247; and an area 244 within the apparatus 250 to contain the disposed parts.

FIGS. 7-7C show the components of the blood collection receptacle 261 in cross sectional views. FIG. 7 shows the blood collection receptacle 261 of FIG. 6A. The receptacle 261 comprises the lancet 267 comprising a needle 268, a blood collection container, such as a capillary tube 255, a sanitizing agent 266 within a dispenser 265, a heater 264, a sensor 269, and a circuit 270 that controls the components.

In FIG. 7A, the finger 10 is placed on the receptacle 261 and a dispenser 265, adapted to dispense a sanitizing agent 266 on a portion of a finger 10 of a patient placed within the insertion port, sprays a sanitizing agent 266 onto the finger 10. In FIG. 7B a cross-sectional view of the finger 10 is pierced by the needle 268 of the lancet 267. The next step, wiping the first drop of blood is not shown within FIGS. 7-7C as the mechanism 259 may be a separate component, and not be a component of the receptacle 261. FIG. 7C shows the blood collection into a capillary tube 255, for collecting a predetermined amount of blood. A heater 264, adapted to warm the finger within the automatic blood collection apparatus, can be utilized to enhance a blood flow of the patient. Also, as shown in FIG. 2A, a massaging mechanism 263 may be used to massage the finger within the automatic blood collection apparatus 200 to enhance a blood flow of the patient. A sensor 269 measures the amount of blood drawn from the patient.

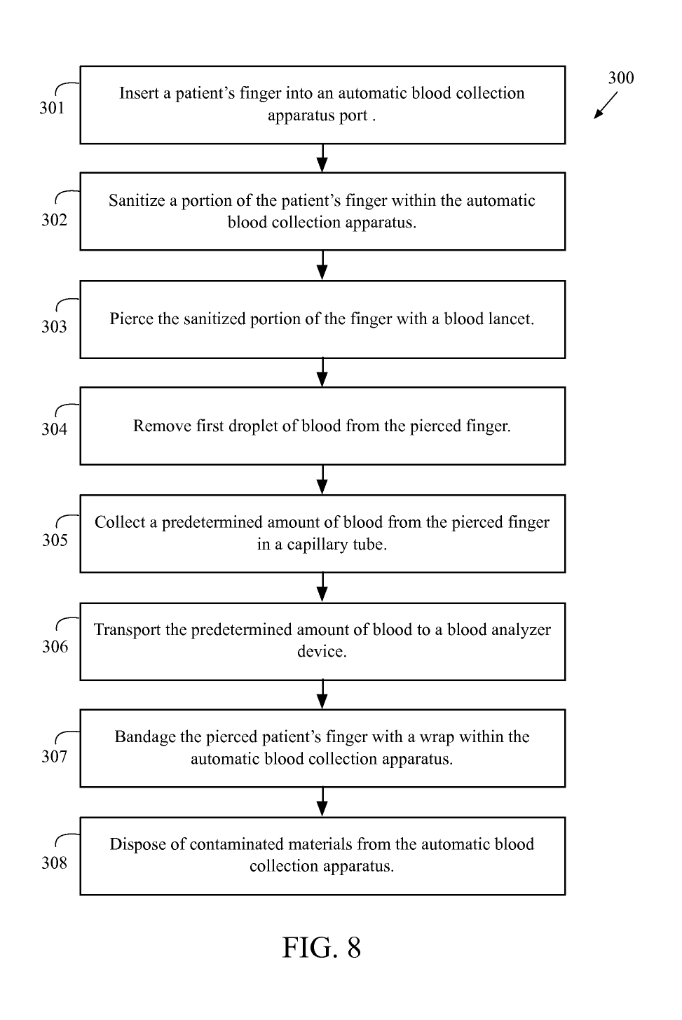

A flow chart of a method 300 for automatic blood collection from a patient is illustrated in FIG. 8. At block 301, a finger of a patient is inserted into a port of an automatic blood collection apparatus. At block 302, a portion of the finger of the patient is sanitized within the automatic blood collection apparatus. At block 303, the sanitized portion of the finger is pierced with a needle of a lancet. At block 304, a first droplet of blood is removed from the pierced finger. At block 305, a predetermined amount of blood is collected from the pierced finger in a capillary tube. At block 306, the predetermined amount of blood is transported to a blood analyzer device. At block 307, the pierced finger of the patient is bandaged with a wrap within the automatic blood collection apparatus. At block 308, contaminated materials from the automatic blood collection apparatus are disposed of in an appropriate manner.

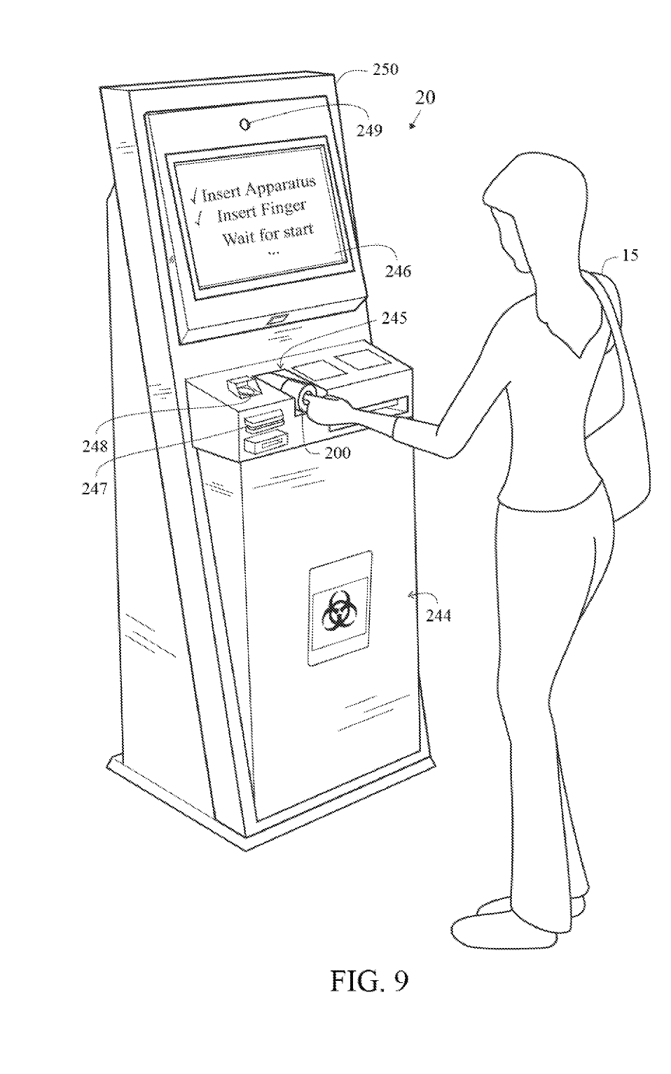

FIG. 9 illustrates the automatic blood collection apparatus being utilized by a user 15. The disposable apparatus 200 is inserted into the controller apparatus 250. The controller apparatus 250 comprises one or more of the following elements: a control display screen 246; a bay 245, including a mechanical lock (not shown), wherein the disposable apparatus 200 is locked into place; biometric identification components, such as a camera 249, a fingerprint reader 248, or an identification card reader 247; and an area 244 within the apparatus 250 to contain the disposable components.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. An apparatus for automatic blood collection from a patient, the apparatus comprising:
a housing having an insertion port;
a lancet comprising a needle, the lancet positioned within the housing and in communication with the insertion port;
a dispenser positioned within the housing, the dispenser comprising a sanitizing agent;
a mechanism for removing an initial droplet of blood, the mechanism positioned within the housing; and
a capillary tube for collecting a predetermined amount of blood, the capillary tube positioned within the housing and in communication with the insertion port;
wherein the dispenser is adapted to dispense the sanitizing agent on a portion of a finger of a patient placed within the insertion port;
wherein the needle is adapted to pierce the sanitized finger;
wherein the mechanism is adapted to remove the initial droplet of blood from the finger;
wherein the capillary is adapted to collect the predetermined amount of blood from the patient.

2. The apparatus according to claim 1 further comprising a transport mechanism for transport the capillary of blood to a blood analyzer.

3. The apparatus according to claim 1 further comprising a heater adapted to warm the finger within the automatic blood collection apparatus to enhance a blood flow of the patient.

4. The apparatus according to claim 1 further comprising a massage mechanism adapted to massage the finger within the automatic blood collection apparatus to enhance a blood flow of the patient.

5. The apparatus according to claim 1 further comprising a vacuum mechanism to withdraw air from the insertion port of the automatic blood collection apparatus to enhance a blood flow of the patient.

6. The apparatus according to claim 1 further comprising a biasing member positioned within the housing and in communication with the insertion port, and adapted to bias the finger of the patient into an optimal position within the automatic blood collection apparatus.

7. The apparatus according to claim 1 further comprising a sensor for measuring the amount of blood drawn from the patient, the sensor positioned within the housing.

8. The apparatus according to claim 1 further comprising at least one of variable vacuum mechanism, a massage mechanism and a heating mechanism adapted to optimize collection of blood from the patient with minimal bleeding by the patient.

9. The apparatus according to claim 1 further comprising a blood collection receptacle in flow communication with the capillary tube for receiving and collecting the blood from the patient.

10. The apparatus according to claim 9 further comprising an ejection mechanism for ejecting the blood collection receptacle from the housing.

11. The apparatus according to claim 1 further comprising a dressing dispenser located at an opening of the insertion port, the dressing dispenser comprising a dressing, wherein the dressing dispenser is adapted to wrap the dressing around the finger of the patient, wherein the dressing protects and avoids cross contamination of the finger.

12. A method for automatic blood collection from a patient, the method comprising:
inserting a finger of a patient into a port of an automatic blood collection apparatus;
sanitizing a portion of the finger of the patient within the automatic blood collection apparatus;
piercing the sanitized portion of the finger with a needle of a lancet;
removing a first droplet of blood from the finger;
collecting a predetermined amount of blood from the finger in a capillary tube;
transporting the predetermined amount of blood to a blood analyzer device; and
disposing of a plurality of contaminated materials from the automatic blood collection apparatus.

13. The method according to claim 12 further comprising warming the finger within the automatic blood collection apparatus to enhance a blood flow of the patient.

14. The method according to claim 12 further comprising massaging the finger within the automatic blood collection apparatus to enhance a blood flow of the patient.

15. The method according to claim 12 further comprising applying a vacuum to the port of the automatic blood collection apparatus to enhance a blood flow of the patient.

16. The method according to claim 12 further comprising biasing the finger of the patient into an optimal position within the automatic blood collection apparatus.

17. The method according to claim 12 further comprising measuring the amount of blood drawn from the patient utilizing a sensor within the automatic blood collection apparatus.

18. The method according to claim 12 wherein the plurality of contaminated materials comprises the needle and a wipe utilized to remove the first droplet of blood.

19. The method according to claim 12 further comprising dressing the finger of the patient with a wrap within the automatic blood collection apparatus, wherein the dressing protects and avoids cross contamination of the finger.

20. An apparatus for dressing a punctured finger of a patient subsequent to automatic blood collection from a patient, the apparatus comprising:
a housing having an insertion port;
a dressing dispenser located at an opening of the insertion port, the dressing dispenser comprising a dressing, wherein the dressing dispenser is adapted to automatically wrap the dressing around a finger of the patient as the finger is removed from the insertion port;
wherein the dressing protects and avoids cross contamination of the finger.
